(19) United States
(12) Patent Application Publication
(10) Pub. No.: US 2004/0049141 A1
(43) Pub. Date: Mar. 11, 2004

(54) Universal wrist splint with removable dorsal stay

(76) Inventors: E. Gerald Slautterback, Coral Springs, FL (US); Rhonda Machin, Weston, FL (US); Jody Brown, Weston, FL (US)

Correspondence Address: DOUGHERTY, CLEMENTS & HOFER, 1901 ROXBOROUGH ROAD, SUITE300, CHARLOTTE, NC, 28211, US

(21) Appl. No.: 10/294,929

(22) Filed: Nov. 14, 2002

Related U.S. Application Data
(63) Continuation-in-part of application No. 29/167,220, filed on Sep. 11, 2002, now Pat. No. D477088.

(51) Int. Cl. A61F 5/00

(52) U.S. Cl. 602/21

(57) ABSTRACT

An universal wrist support device that can be used for the prevention of injury and for the rehabilitation of various wrist conditions so that multiple wrist splints/braces are not required. The support device includes a soft, flexible closure for wrapping around the wrist of a wearer, a pair of palmar stays for supporting the wrist at about a fifteen degree angle, a removable dorsal support structure for immobilizing the wrist, and straps for tightening and securing the wrist support device to the wearer. When used with the dorsal support structure, the device serves as a “splint”, providing maximum support and immobilization of the wrist, and preventing flexion and extension of the wrist. The splint configuration of the wrist support device is used to support a moderately to severely weakened or injured wrist due to strains or sprains, or to assist in the healing process after surgery or after cast removal. When the dorsal support structure is removed from the wrist support device, the device serves as a “brace” for supporting and rehabilitating mildly to moderately weakened or injured wrists. The brace refrains the wrist from moving in flexion while allowing extension, and allows freedom of movement of the digits of the hand. The brace also supports the wrist in a neutral cock-up positioned to provide relief from the symptoms of carpal tunnel syndrome. The wrist support device is universally configured to interchangeably fit left and right hands and is adjustable in size to accommodate different wearers.

### CROSS-REFERENCE TO RELATED APPLICATIONS

This Application is a Continuation-in-Part of U.S. Design Patent Application Serial No. 29/167,220, filed Sep. 10, 2002.

### FIELD OF THE INVENTION

The present invention relates generally to orthopedic devices for supporting and stabilizing a human wrist, and more particularly to an universal wrist splint with a removable dorsal stay for treating and preventing conditions of the wrist.

### BACKGROUND OF THE INVENTION

A human forearm has a skeleton structure made up of a lateral bone called the radius and a medial bone called the ulna, both of which contribute to the movement of the hand and wrist. Eight carpal bones form a carpal “tunnel”, located between the forearm and the hand, in the wrist to allow for the various motions of the wrist. A total of twenty-seven bones, joints and soft tissues such as muscles, ligaments and tendons make up the hand and wrist.

Wrist motion is controlled by muscles and tendons located at the forearm with stability provided by the transverse carpal ligament located across the carpus. Furthermore, the fingers and thumb are straightened and the hand is raised by the help of the extensor tendons while flexor tendons help bend the fingers, thumb and hand. These tendons pass through the carpal tunnel channel and are surrounded, protected and guided by tendon sheaths. The median nerve also passes through the carpal tunnel channel which supplies feeling to the thumb, index and middle fingers.

Because the wrist is anatomically complex, it is subject to several types of injuries or diseases that weaken the wrist. One such disease is arthritis which slowly destroys the joint causing inflammation and stiffening. Arthritis can advance to osteoarthritis or degenerative joint disease that breaks down the actual bones of the joint. A person afflicted with arthritis may require supplemental support for the weakened wrist joint.

Wrist injuries may occur in numerous ways. For example, sprains and strains can occur due to overuse or repetitive use of the hand or wrist. A strain is an inflammation of the tendons that connect muscles to the bones causing tenderness, pain and mild swelling. If left untreated, the strained tendon can start to pull away for the muscle with slight tearing progressing to tendinitis. A wrist sprain is a stretching or tearing of a ligament group (versus a tendon as in a strain) in the wrist or hand. Symptoms may include pain, swelling and even loss of function. In either case, external support is often necessary to allow the strained muscle area or sprained ligament group to be rested and allowed to heal.

Another common injury to a wrist is a contusion. A wrist contusion is the result of a direct blow to a structure of the wrist, causing tenderness, decreased range of motion, sprain, and possibly a wrist fracture. Forced hyperextension resulting from outstretching an arm to break a fall is the most common cause of wrist fracture, particularly a fracture to the radius or scaphoid of the carpus bones. Conditions of the wrist caused by contusion may require auxiliary stability and support to be provided to the wrist to facilitate healing. Where a traumatic injury has occurred to the wrist, such as a fracture, upon cast removal, the weakened wrist will often require additional stabilization during the healing and physical therapy process.

The wrist may also be affected by cumulative trauma disorder. Cumulative trauma disorders are physical problems affecting joints and soft tissues (muscles, tendons and nerves). This condition is marked by discomfort, swelling and muscle fatigue and can limit the range of motion and reduce the ability to grip objects. Again, the weakened wrist may require support to increase function and aid in the healing process.

Carpal tunnel syndrome is a condition of the hand caused by swelling of the synovial membrane, tendon inflammation, and any condition that reduces the available space in the carpal tunnel, resulting in pressure on the median nerve. The median nerve passes through the “tunnel” formed by eight small carpal bones at the wrist. In particular, the tunnel narrows due to swelling of the protective sheaths around the wrist tendons, resulting in pressure on the median nerve. Most commonly, the narrowing occurs when a dense fibrous tissue called the transverse carpal ligament forms over and compresses the median nerve. Carpal tunnel syndrome can also occur from trauma such as blunt contusions, carpal bone dislocation or fracture which compromises the carpal tunnel.

Activities that cause flexion of the wrist and fingers, such as repetitive and forceful grasping of the hand and repetitive bending of the wrist, are common causes of carpal tunnel syndrome. Carpal tunnel syndrome is a particular problem for workers in industries which require repeated manual hand motions, such as modern day keyboard operation. Common symptoms of this condition include numbness, tingling and pain in the hand. Without treatment, the condition can worsen, causing the hand to become weak, to lose muscle control and eventually to become crippled.

Numerous prior art devices are available to support and rehabilitate conditions of the wrist. The various devices are selected according to the specific condition and the severity of that condition. For example, elastic pullover wristlets and neoprene wrist wraps are used to treat mild strains or contusions. Where additional support is needed for the wrist, such as for moderate strains and sprains, the support device typically includes a stiffening member to partially restrain movement of the wrist. In cases of severe strains, after surgery, or after cast removal, a splint having a plurality of stiffening members is desired to support and immobilize the wrist. Each of these conditions require a different wrist support. Moreover, as the injured wrist heals, separate wrist support devices may be required to facilitate rehabilitation by allowing increasing degrees of function.

Still other devices are currently available for the prevention, alleviation of discomfort, or treatment of carpal tunnel syndrome, and to allow continued activity when possible. Such wrist devices include bracelets, gloves and wrist braces/splints, and are configured to open the carpal to relieve pressure from the median nerve. Bracelets create pressure points around the wrist to alleviate carpal tunnel syndrome. Pressure on these pressure points can be uncomfortable to the wearer. Gloves are configured to provide protection from vibrations and shocks which can cause carpal tunnel syndrome. However, gloves are not suitable for treatment of carpal tunnel syndrome caused by other activities, such as keyboard operation. Traditional wrist braces/splints are limited in that they are adapted solely for either the left or right hand, may unnecessarily restrict hand mobility, may be uncomfortable to wear, and may not provide adequate support.

Moreover, in regard to carpal tunnel syndrome caused by keyboard operation, it is desirous that the wrist device support the wrist at about a 15 degree cock-up angle, referred to as the neutral position, to allow the person maximum relief during keyboard operation. Bracelets, gloves, and conventional braces/splints do not support the wrist in the neutral position.

Accordingly, what is needed is a wrist support device that provides support and allows rehabilitation of a variety of conditions of a wrist, from mild to traumatic injuries. Also needed is a wrist support device that is suitable for use throughout the various stages of recovery from a wrist injury. Moreover, a wrist support device is needed that prevents occupational injuries such as carpal tunnel syndrome without unnecessarily impeding freedom of movement of a wearer's hand and digits of the hand. Further needed is a wrist support device that can be interchangeably used on either the left hand or right hand. Finally, it is desired that a single wrist support device fulfill all of these needs.

### SUMMARY OF THE INVENTION

The present invention is a wrist support device that can be used of the prevention of injury and for the rehabilitation of various wrist conditions so that multiple splints/braces are not required. Moreover, the same wrist support device is suitable for use throughout the various stages of rehabilitation, and is universally configured to interchangeable fit left and right hands, again necessitating only one wrist support device.

The device includes a removable dorsal support structure which allows the device to serve as a “splint” when the dorsal structure is attached, or as a “brace” when the dorsal structure is detached. As a splint, the device provides maximum support and immobilization of the wrist, preventing flexion and extension of the wrist. The splint configuration of the device is used to support a moderately to severely weakened or injured wrist, due to strains or sprains, or to assist in the healing process after surgery or after cast removal.

When used as a brace, the wrist support device is used for supporting and rehabilitating mildly to moderately weakened or injured wrists. The brace restrains the wrist from moving in flexion while allowing extension, and allows freedom of movement of the digits of the hand. The brace also supports the wrist in a neutral cock-up position to provide relief from the symptoms of carpal tunnel syndrome.

In the broadest sense, the wrist support device relates to a wrist support device that includes a body for enclosing a wrist of a wearer. The body is supported by a palmar stay and a removable dorsal stay. Preferably, the palmar stay supports the wrist at about a 15 degree cock-up angle, referred to as the neutral angle.

In its broadest sense, the invented wrist support device also encompasses a wrist support device that includes a flexible closure having a distal section terminating along a wearer's hand and a proximal section terminating along the wearer's forearm. The closure includes a continuous opening for allowing a wrist to be placed within the closure. The device further includes a support structure that can be removed from and attached to the top of the closure in such manner that the wearer's wrist is restricted from moving in extension. A palmar stay is disposed on the bottom side of the closure to restrict movement of the wearer's wrist in flexion. Means are provided for securing the closure to the wearer. Preferably, the support structure includes a dorsal stay that has a distal end that terminates along the wearer's forearm.

### OBJECTS OF THE INVENTION

The principle object of the present invention is to provide a device that supports an injured wrist and facilitates rehabilitation of the wrist.

Another object of this invention is to provide a device suitable for the treatment of a wide range of wrist conditions so that multiple wrist splints, braces or other wrist devices are not needed.

Another object of this invention is to provide a single device that can be used throughout the various stages of rehabilitation.

A further object of this invention is to provide a device for both the prevention and treatment of carpal tunnel syndrome.

Another object of this invention is to provide a device for supporting the wrist and hand in a 15 degree cock-up position, from the horizontal, referred to as the neutral position.

Another object of this invention is to provide a device that can be interchangeably worn on the left or right hand, and on a wide range of hand sizes.

A further object of this invention is to provide a wrist support device that is comfortable to wear.

### BRIEF DESCRIPTION OF THE DRAWINGS

The foregoing and other objects will become more readily apparent by referring to the following detailed description and to the appended drawings in which:

FIG. 1 is a perspective view of the invented wrist support device, attached to the right wrist of a wearer, and being used as a splint;

FIG. 2 is an exploded perspective view of the wrist support device of FIG. 1, with a dorsal support structure detached therefrom so that the wrist support device functions as a brace, and with dorsal and palmer stays exploded from the wrist support device for illustrative purposes;

FIG. 3 is a perspective view of the wrist support device of FIG. 1, but with the device attached to the left wrist of the wearer;

FIG. 4 is a bottom view of the wrist support device of FIG. 1;

FIG. 5 is a front end view of the wrist support device of FIG. 1; and

FIG. 6 is a rear end view of the wrist support device of FIG. 1.

### DETAILED DESCRIPTION

The present invention is a device for supporting a wrist in order to prevent injury and to facilitate rehabilitation of an injured wrist. The wrist support device is suitable for treatment of a wide range of wrist conditions so that multiple splints, braces or other wrist devices are not needed. Moreover, the same wrist support device is suitable for use throughout the various stages of rehabilitation, again necessitating only one wrist support device.

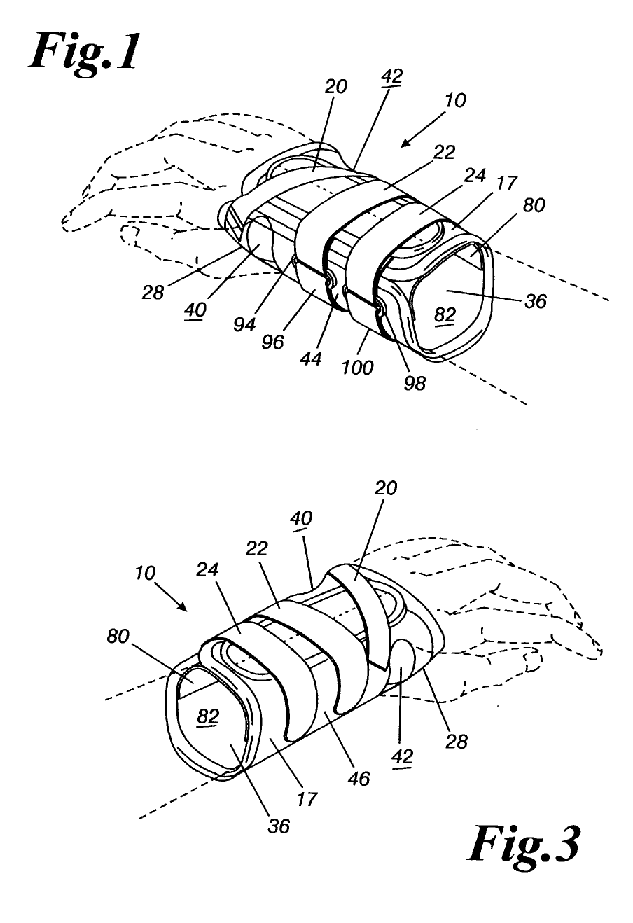

Referring to the drawings, and particularly to FIGS. 1 and 2, the invented wrist support device 10 is shown in position enclosing a wearer's wrist. The invented wrist support device 10 has a pair of palmar stays 14, 15 (FIG. 2), and a removable dorsal support structure 16 to provide a selectable amount of support. When used with the dorsal support structure 16, as illustrated in FIG. 1, the device 10 serves as a splint, providing maximum support and immobilization of the wrist to prevent flexion and extension of the wrist. The splint configuration of the wrist support device 10 is used to support a moderately or severely weakened or injured wrist due to strains or sprains, and to assist in the healing process after surgery or after cast removal.

As illustrated in FIG. 2, with the dorsal support structure 16 removed, the wrist support device 10 serves as a brace. (The palmer stays 14, 15 and dorsal stays 66, 68 are exploded for illustrative purposes and are further described below). The brace is suitable for treating mildly to moderately weakened or injured wrists by restraining the wrist from moving in flexion while allowing extension of the wrist and freedom of movement of the digits of the hand. The brace also supports the wrist in a neutral cock-up position to provide relief from the symptoms of carpal tunnel syndrome. The wrist support device 10 is universally configured to interchangeably fit left or right hands and is adjustable in size to accommodate different wearers.

Referring to FIG. 2, the wrist support device 10 comprises a soft, flexible closure 17 for wrapping around the wrist of a wearer, the pair of palmer stays 14, 15 for supporting the wrist at about a fifteen degree angle, referred to as the neutral cock-up position, the removable dorsal support structure 16 for optionally immobilizing the wrist, and straps 20, 22, 24 for tightening and securing the wrist support device 10 to the wearer.

The soft, flexible closure 17 is adapted to wrap around the wrist with the proximal portion 26 extending along the wearer's forearm and the distal portion 28 extending along the hand, preferably terminating prior to the knuckles and palmar fold of the hand to allow unrestricted movement of the digits of the hand. The closure 17 has a continuous opening 30 extending the full length of the closure 17 so that the closure 17 can be opened apart and a wrist inserted therein. Two opposing edges 32, 34 of the closure 17 are generally juxtaposed when the wrist support device 10 is properly attached to the wrist. However, to accommodate wrists of different girth, the device 10 properly functions with the edges 32, 34 slightly spaced apart or when one edge 34 overlaps the opposite side of the closure 17.

Although there are many suitable materials from which the closure 17 may be formed, it is preferred that the closure is made of Tripermolim to provide comfort, durability and resiliency. Tripermolim is a tri-laminate which includes an inner layer 36 made of a Lycra™ spandex and polyester blend to provide a soft feel against the wearer's skin, an ester-type foam middle layer (not shown) to provide a resilient cushion between the wearer and the stays and/or the environment in general, and an outer layer 38 made out of a Lycra™ spandex and nylon blend having unbroken loop construction. The unbroken loop construction allows the straps 20, 22, 24, which have complementary Velco™-type hook and pile fasteners, to releaseably attach to the closure 17 for tightening and securing the wrist support device 10 to the wearer.

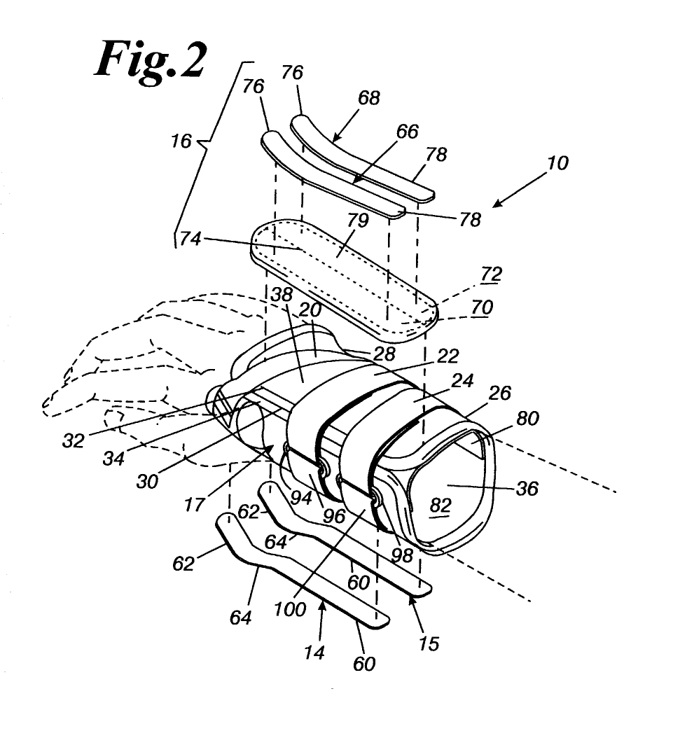

As illustrated by FIGS. 1 and 3, right and left thumb openings 40, 42 are respectively provided in right and left sides 44, 46 of the distal portion 28 of the closure 17. The openings 40, 42 are sized to receive a thumb and allow the wrist support device 10 to be interchangeably worn on the left or right hand.

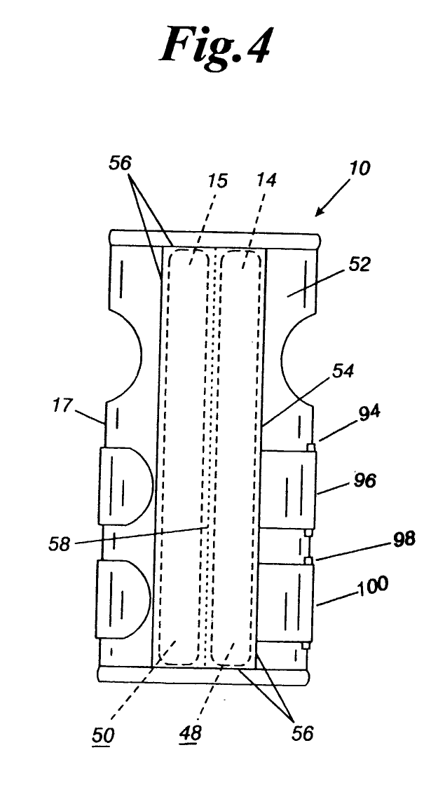

Referring to FIG. 4, left and right sleeves 48, 50 are provided on the bottom surface 52 of the closure 17 for housing the pair of palmar stays 14, 15. Preferably, the sleeves 48, 50 are formed by a durable, tightly stitched fabric 54, affixed to the closure 17 by suitable means, such as perimeter stitching 56, with a longitudinal centerline of stitching 58 that divides the left sleeve 48 from the right sleeve 50. The sleeves 48, 50 extend about the full length of the wrist support device 10, enclose the palmar stays 14, 15 and define their position.

The left and right palmar stays 14, 15 are positioned on opposed sides of the longitudinal centerline 58 and have widths suitable to restrict opposition between the thumb and fifth digit. That is, one stay provides support at or near the center of the hypothenar eminence while the other stay provides support at or near the center of the thenar eminence so that movement between the hypothenar eminence and thenar eminence is limited and/or restricted.

Referring to FIG. 2, the palmar stays 14, 15 are also preferably configured to restrain the wrist from moving in flexion, and to maintain the wrist at about the neutral fifteen-degree “cock-up” angle. That is, each stay 14, 15 has a generally linear proximal section 60 extending a predetermined length along the forearm of the wearer, and an inclined distal section 62 supporting the wrist and heel of the hand in the neutral position. More preferably, each stay further includes a declined intermediate section 64 to accommodate the heel of the hand.

Although two stays 14, 15 are preferred, to allow the stays 14, 15 to independently “float” according to muscle and tendon movement, other stay arrangements may also be used. For example, a single wide palmar stay may be used. As another example, a split stay having a wide distal section provides suitable support for the wrist and hand and a pair of proximal leg sections extending from the distal section, along the forearm, and allows a degree of “floating”. In each case, suitable sleeve(s) are configured to accommodate the various stay shapes.

The palmar stays 14, 15 are made of any suitable material that provides sufficient support to the wrist. That is, the stays 14, 15 may be rigid to semi-rigid and may resiliently bend without breaking. Common materials from which the stays 14, 15 may be made include plastic, aluminum, steel, and carbon steel.

Where greater support is needed, the dorsal support structure 16 is attached to the top of the closure 17 (FIG. 1) to provide maximum support and to immobilize the wrist. The dorsal support structure 16 extends nearly the length of the wrist support device 10 and provides housing for left and right dorsal stays 66, 68. In particular, the dorsal support structure 16 defines left and right sleeves 70, 72 into which the dorsal stays 66, 68 are disposed. A longitudinal centerline of stitching 74 separate the left and right dorsal stays 66, 68 into their respective sleeves 70, 72.

The dorsal stays 66, 68 have a sufficient width and length to support and generally immobilize the wrist from moving in extension. In particular, the dorsal stays 66, 68 extend across the top of the wrist with the distal section 76 of each stay 66, 68 extending over the top of the hand, terminating near the knuckles, and the proximal section 78 of each stay 66, 68 extending over the forearm. The distal end 76 of each dorsal stay 66, 68 is preferably slightly arcuate upwards to cooperate with the palmar stays 14, 15 in positioning the wrist and hand in the neutral “cock-up” position. As with the palmar stays 14, 15, the use of left and right dorsal stays 66, 68 allow the stays 66, 68 to independently “float” to accommodate movement of tendons and the like. The dorsal stays 66, 68 may be rigid, semi-rigid, or resilient, and may be made of any suitable material that provides the necessary degree of support and immobilization to the wrist, such as plastic, aluminum, carbon steel and steel. Other dorsal stay configurations may also be used. For example, a single wide dorsal stay may be used instead of the pair of dorsal stays. As another example, a split stay may be used.

The dorsal support structure 16 can be made of any suitable material, but preferably the top surface 78 includes a Lycra™ spandex and nylon blend having an unbroken loop construction to which the straps 20, 22, 24 can be releasebly attached, to tighten and secure the wrist support device 10 to the wearer. The bottom surface of the dorsal support structure 16 includes Velcro™-type hook fasteners (not shown) to allow the dorsal support structure 10 to simply and quickly be attached, and removed from, the closure 17.

Advantageously, since the dorsal support structure 16 can be selectively attached and removed, the wrist support device 10 can be used to treat a variety of wrist conditions. First, when the wrist support device 10 is used without the dorsal support structure 16, the device 10 performs as a brace for the treatment of mild strains and sprains, and supports the wrist in the neutral position for the treatment of carpal tunnel syndrome. Second, when the dorsal support structure 16 is applied to the wrist support device 10, the device serves as a splint for the treatment of moderate to severe strains and sprains, and for use after surgery and after cast removal.

It is noted that the dorsal support structure 16 can be attached to the closure 17 by any suitable means. As one example, complementary snaps can be provided on the dorsal support structure and closure to allow releaseable attachment there-between. However, since snaps can cause pressure points and ridge lines, this means of attachment is not preferred.

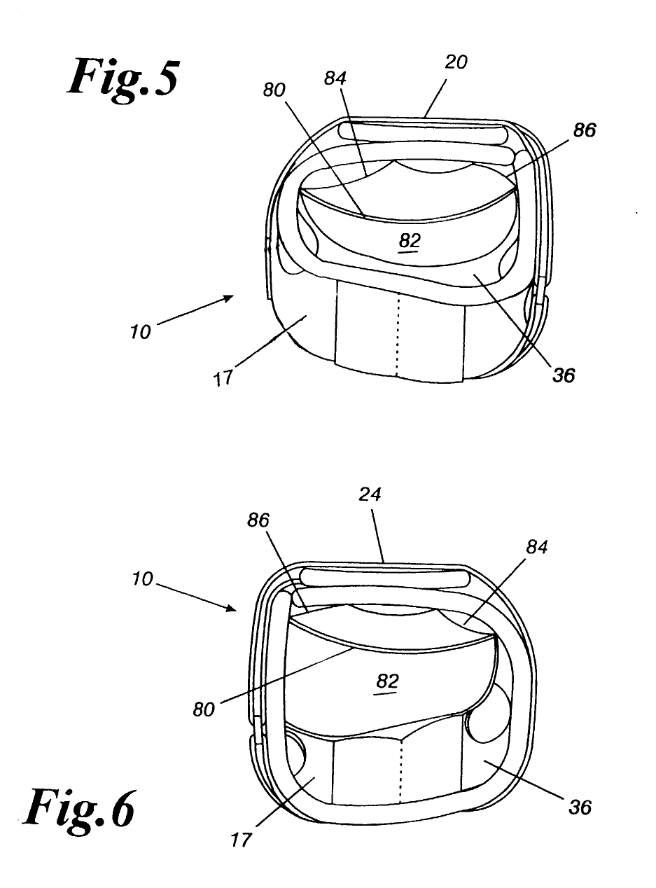

Referring to FIGS. 1-3, 5 and 6, the closure 17 and an elastic fabric 80 form a longitudinally oriented sleeve 82 for receiving a wrist. The sleeve 82 holds the wrist support device 10 in place on the wrist, allowing the wearer to adjust the straps 20, 22, 24 with one hand, to tighten and secure the wrist device 10. The closure 17 forms the sides and bottom of the sleeve 82. The elastic fabric 80 forms the top of the sleeve and is attached to the closure 17 by having its opposed left and right ends 84, 86 stitched to the closure 17, as shown in FIGS. 5 and 6. The elastic character of the fabric 80 allows for the wearer's hand to be inserted through the sleeve 82, accommodates wrists of different girth, and resiliently tightens to the wearer so that the wearer can tighten the device 10 to his wrist by using only one hand.

Referring to FIG. 1, a plurality of straps 20, 22, 24 are used to tighten the wrist support device 10 to the wrist and to provide compression control. The distal strap 20 is affixed at one end to the left side of the closure 17, above the right thumb opening 40, with the opposed end extending clockwise over the closure 17 and attaching to the closure 17 and dorsal stay structure 16. Preferably, the distal strap 20 angles toward the proximal end of the wrist support device 10 to provide secure tightening of the distal closure portion 28 to the hand.

Referring collectively to FIGS. 1 and 2, the intermediate strap 22 is affixed at one end to the top of the closure 17, with the opposed end extending counterclockwise over the closure 17, passed through a ring 94, tensioned to the wrist an appropriate amount, then folded upon itself and across the closure 17 so that opposing hook and loop fasteners frictionally mate to hold the set amount of tension. The ring 94 is held in a looped strap 96 which is affixed to the bottom of the closure 17 (See also FIG. 4).

The proximal strap 24 is arranged similarly to the intermediate strap 22. That is, the proximal strap 24 is affixed at one end to the top of the closure 17, with the opposed end extending counterclockwise over the closure 17, passed through a ring 98, tensioned to the forearm an appropriate amount, then folded upon itself and across the closure 17 so that opposing hook and look fasteners frictionally mate to hold the set amount of tension. The ring 98 is held in a looped strap 100 which is affixed to the bottom of the closure 17 (See also FIG. 4).

As it will be appreciated by those skilled in the art, other strap arrangements are possible for tensioning the wrist support device 10 to a wearer. Moreover, other means for tensioning the wrist support device may be used, such as straps which tie around the device, straps provided with length adjustment ties, and straps with complementary buckle and tongue components.

In use, the invented wrist support device 10 supports the wrist in order to prevent further injury to the wrist and to facilitate rehabilitation of the wrist. The device 10 is suitable for treatment of a wide range of wrist conditions, from mild strains and sprains to traumatic wrist injuries.

Referring to FIG. 2, the wrist support device 10 serves as a “brace” when used without the dorsal support structure 16. The brace is ideal for use in the treatment of minor or mild strains and sprains, and supports the wrist in the neutral position for the treatment of carpal tunnel syndrome. By having the dorsal support structure 16 removed from the device 10, the wearer maintains freedom of movement in flexion which allows the wearer good use of his hand, and sufficient movement to strengthen the wrist.

Referring to FIG. 1, when the dorsal support structure 16 is attached to the wrist support device 10, the device 10 serves as a “splint”, supporting and immobilizing the wrist. The splint is well-suited when greater immobilization is require to the wrist area, such as for treatment of mild to severe strains and sprains, after surgery, and after cast removal.

To attach the wrist support device 10 to a wearer's wrist, the wearer inserts either their left or right hand through the sleeve 82 formed by the closure 17 and fabric 80. The wearer selects whether the dorsal support structure 16 should be used with, or removed from, the wrist support device 10, depending on the type and severity of the injury. Thereafter, the wearer tightens the straps 20, 22, 24 and attaches the straps 20, 22, 24 to the outer layer 38 of the closure 17. When the dorsal support structure 16 is attached to the wrist support device 10, the straps 20, 22, 24 also attach to the dorsal support structure 16 by Velcro™-type hook and loop fasteners.

To remove the wrist support device 10 from the wearer's wrist, the straps 20, 22, 24 are simply detached from the closure 17 and the wrist support device 10 removed from the wrist.

### Summary of the Achievement of the Objects of the Invention

From the foregoing, it is readily apparent that we have invented a wrist support device that can be used for both the prevention of injury and the rehabilitation of various wrist conditions so that multiple wrist splints/braces are not required. In particular, the wrist support device can be used as a splint to support and immobilize the wrist, or can be used as a brace, by detaching a dorsal support structure from the device, for rehabilitation of minor injuries or for the treatment of carpal tunnel syndrom. When used as a brace, the device supports the wrist in a neutral cock-up position while allowing the wrist to move in extension and allowing ample freedom of movement of the digits of the hand. Moreover, the device is universal, in that it is configured for use with either the left or right hands.

It is to be understood that the foregoing description and specific embodiments are merely illustrative of the best mode of the invention and the principles thereof, and that various modifications and additions may be made to the apparatus by those skilled in the art, without deviating from the spirit and scope of this invention, which is therefore understood to be limited only by the scope of the appended claims.

What is claimed is:

1. A wrist support device, comprising:
a body, for enclosing a wrist of a wearer, said body having a distal portion and a proximal portion;
a palmar stay supporting said body; and
a dorsal stay removably attachable to said body.

2. The wrist support device according to claim 1, wherein at least a portion of said dorsal stay is arcuate.

3. The wrist support device according to claim 1, wherein said dorsal stay comprises two dorsal stays.

4. The wrist support device according to claim 1, wherein said palmar stay has a distal section positional along the hand of a wearer, an intermediate section positional along the heal of the hand, and a proximal section positional along the forearm of the wearer, and wherein said stay is adapted to support the hand in about a 15 degree neutral position.

5. The wrist support device according to claim 4, wherein the proximal section of said palmar stay is generally linear and the distal section of said palmar stay is angled in relation to the proximal section of the palmar stay.

6. The wrist support device according to claim 5 wherein said palmar stay comprises two palmar stays.

7. The wrist support device according to claim 1, wherein said dorsal stay is enclosed within a structure, and wherein said structure is removably attachable to said body.

8. The wrist support device according to claim 7, wherein said structure is removably attachable to said body by hook and loop fasteners.

9. The wrist support device according to claim 8, further comprising means for tightening said body to the wearer, and wherein at least a portion of said tightening means is releaseably attachable to said body and to said structure.

10. The wrist support device according to claim 8, wherein said body includes a first opening capable of receiving a right thumb and a second opening capable of receiving a left thumb such that the wrist support device may interchangeably be worn on the left and right hands.

11. The wrist support device according to claim 8 wherein said tightening means includes a distal strap for tightening the wrist support device to the hand of the wearer, an intermediate strap for tightening the wrist support device to the wrist of the wearer, and a proximal strap for tightening the wrist support device to the forearm of the wearer.

12. The wrist support device according to claim 1, wherein said body includes a continuous opening extending the full length of the wrist support device, so that the wrist support device can be opened to allow the wearer's wrist to be placed within the wrist support device.

13. The wrist support device according to claim 12, further including a member attached to the inner surface of said body, wherein said body and said member collectively form a sleeve capable of enclosing the wrist of the wearer.

14. The wrist support device according to claim 13, wherein said body has an exterior that includes unbroken loop material.

15. A wrist support device, capable of serving as a splint and as a brace, comprising:
a flexible closure having a distal section terminating along a wearer's hand and a proximal section terminating along a wearer's forearm;
said closure having a continuous opening for allowing a wrist to be placed within said closure;
a support structure attachable to and removable from the top of said closure, said support structure is being configured to restrict a wearer's wrist from moving in extension;
a palmar stay disposed on the bottom side of said closure to restrict movement of the wearer's wrist in flexion; and
means for securing the closure to the wearer;
wherein the wrist is generally immobilized when said support structure is attached to said closure, and wherein the wrist can move in flexion when said support structure is detached from said closure.

16. The wrist support device according to claim 15, wherein said support structure includes a dorsal stay having a distal end that terminates prior to the wearer's knuckles and a proximal end that terminates along the wearer's forearm when the wrist support device is properly attached to the wearer.

17. The wrist support device according to claim 16, wherein said support structure includes a dorsal sleeve, and wherein said dorsal stay is disposed within said dorsal sleeve.

18. The wrist support device according to claim 17, wherein said securing means is a plurality of straps, and wherein said support structure releaseably attaches to said closure by hook and loop fasteners.

19. The wrist support device according to claim 18 wherein said plurality of straps are releaseably attachable to said support structure.

20. The wrist support structure according to claim 19, further including an elastic member, wherein said member and closure collectively from a sleeve capable of enclosing the wearer's wrist.
